(19) United States
(12) Patent Application Publication
(10) Pub. No.: US 2013/0072798 A1
(43) Pub. Date: Mar. 21, 2013

(54) OBJECT INFORMATION ACQUIRING APPARATUS AND CONTROL METHOD THEREOF

(71) Applicant: Jiro Tateyama, Yokohama-shi (JP)

(72) Inventor: Jiro Tateyama, Yokohama-shi (JP)

(73) Assignee: CANON KABUSHIKI KAISHA, Tokyo (JP)

(21) Appl. No.: 13/609,454

(22) Filed: Sep. 11, 2012

(30) Foreign Application Priority Data
Sep. 15, 2011 (JP) 2011-201931
Aug. 8, 2012 (JP) 2012-175738

(51) Int. Cl. A61B 8/14

(52) U.S. Cl. 600/444

(57) ABSTRACT

An object information acquiring apparatus includes: a probe that converts an acoustic wave from an object into an electric signal; a unit that moves the probe; a generating unit that generates a plurality of first image data corresponding to tomographs of the object using a plurality of acoustic signals from respective positions of the object interior, and generates second image data using the plurality of acoustic signals; and a display control unit into which the first image data and second image data are input, and which displays on a display unit an image of the object interior, wherein the display control unit displays on the display unit a display based on the first image data, and switches the display when the second image data are input from an identical position of the object.

### BACKGROUND OF THE INVENTION

1. Field of the Invention

The present invention relates to an object information acquiring apparatus and a control method thereof.

2. Description of the Related Art

A conventional ultrasound diagnostic apparatus used for medical image diagnoses employs an ultrasound probe including transducers having an ultrasound wave transmission/reception function. When an ultrasound beam formed from a synthesized wave of ultrasound waves is transmitted toward an object from the ultrasound probe, the ultrasound beam is reflected in an area of the object interior where acoustic impedance varies, or in other words a tissue boundary. By receiving an echo signal generated by the reflection and reconstructing an image on the basis of an intensity of the echo signal, a tissue condition in the object interior can be reproduced on screen as an ultrasound echo image.

Japanese Patent Application Publication No. 2009-28366 (Patent Literature 1: PTL 1) discloses a method of obtaining a three-dimensional ultrasound image of a wide area by performing a mechanical scanning operation using an ultrasound probe. More specifically, in this method, an ultrasound image is obtained while continuously moving a linear array probe in a direction (to be referred to hereafter as an elevation direction) which is orthogonal to and intersects an element array direction (to be referred to hereafter as a lateral direction). The linear array probe is capable of reconstructing a single tomographic slice image by performing electronic scanning using an ultrasound beam. Hence, by overlapping tomographic slice images created in respective positions in the elevation direction, a three-dimensional ultrasound image of an entire mechanical scanning area can be obtained. This three-dimensional image acquiring method is advantaged in terms of both speed and cost.

Japanese Patent Application Publication No. 2010-183979 (Patent Literature 2: PTL 2), meanwhile, discloses means for improving a resolution of ultrasound imaging using adaptive signal processing. A CAPON method, for example, is a type of adaptive signal processing using a spatial averaging method, which is employed in the radar field. The CAPON method serving as a type of adaptive signal processing may also be combined with a frequency domain interferometry (FDI) method. When adaptive signal processing is used, a frequency spectrum of a reception signal received during ultrasound imaging can be flattened with a high degree of precision, and as a result, an ultrasound image having a greatly improved spatial resolution in comparison with a conventional image can be obtained.
PTL 1: Japanese Patent Application Publication No. 2009-28366
PTL 2: Japanese Patent Application Publication No. 2010-183979

### SUMMARY OF THE INVENTION

In the conventional example described in Japanese Patent Application Publication No. 2009-28366, however, on the single tomographic slice image reconstructed by performing electronic scanning with an ultrasound beam using the linear array probe, the image resolution in the elevation direction is much poorer than the image resolution in the lateral direction.

A first reason for this is that a pixel density in the elevation direction must be reduced to a certain extent. By reducing a scanning speed of the mechanical scan performed by the probe in order to increase a scanning pitch of the tomographic slice image, the pixel density in the elevation direction can be increased, but in this case, the duration of a physical load on an examinee increases. A second reason is that an effective aperture angle of the linear array probe in the elevation direction is smaller than the aperture angle in the array direction, and therefore a reconstruction resolution in the elevation direction is poor. This problem can be solved to a certain extent by using a two-dimensional array probe, but in this case, a required electrical circuit scale increases due to an increase in a number of transmission/reception elements, making practical application difficult in terms of cost.

The conventional example described in Japanese Patent Application Publication No. 2010-183979 describes means for improving the image resolution in the lateral direction in relation to a single tomographic slice image (a two-dimensional ultrasound image), but when this method is applied to the elevation direction, an increase in a calculation amount occurs. In other words, increases occur in the scale of a required signal processing circuit and an image memory, making practical application to an apparatus difficult in terms of cost. Moreover, when such a signal processing circuit is provided, a large increase in processing time may occur, making real time image display difficult.

The present invention has been designed in consideration of these problems, and an object thereof is to provide an object information acquiring apparatus with which image display speed and image resolution requirements can both be satisfied.

An object information acquiring apparatus according to the present invention is configured as described below.

More specifically, the present invention provides an object information acquiring apparatus comprising:

a probe in which a plurality of elements that receive acoustic waves propagating from an object and convert said acoustic waves into electric signals are arranged in at least a first direction;

a scanning unit that moves said probe in a second direction that intersects said first direction;

a generating unit that determines intensities of said acoustic waves in respective positions of an object interior using said plurality of electric signals, generates a plurality of first image data corresponding to tomographic images of said object in said second direction using a plurality of acoustic signals based on said intensities, and generates second image data using said plurality of acoustic signals; and

a display control unit into which said first image data and said second image data are input, and which displays on a display unit an image representing information relating to said object interior,

wherein said display control unit displays on said display unit a display based on said first image data, and

when said second image data are input from an identical position of said object, switches said display from said display based on said first image data to a display based on said second image data.

Further, the present invention provides an object information acquiring apparatus comprising:

a probe in which a plurality of elements that receive acoustic waves propagating from an object and convert said acoustic waves into electric signals are arranged in at least a first direction;

a scanning unit that moves said probe in a second direction that intersects said first direction;

a generating unit that determines intensities of said acoustic waves in respective positions of an object interior using said plurality of electric signals, generates a plurality of first image data corresponding to tomographic images of said object in said second direction using a plurality of acoustic signals based on said intensities, and generates second image data and third image data using said plurality of acoustic signals; and

a display control unit into which said first, second, and third image data are input, and which displays on a display unit an image representing information relating to said object interior,

wherein said display control unit displays on said display unit a display based on said second image data, and

when said third image data are input from an identical position of said object, switches said display from said display based on said second image data to a display based on said third image data.

Further, the present invention provides a control method for an object information acquiring apparatus having:

a probe in which a plurality of elements that receive acoustic waves propagating from an object and convert said acoustic waves into electric signals are arranged in at least a first direction;

a scanning unit that moves said probe in a second direction that intersects said first direction;

a generating unit that determines intensities of said acoustic waves in respective positions of an object interior from said plurality of electric signals, and generates image data using a plurality of acoustic signals based on said intensities; and

a display unit that displays an image of said object based on said image data,

the control method comprising the steps of:

generating by said generating unit a plurality of first image data corresponding to tomographic images of said object in said second direction;

displaying on said display unit a display based on said first image data;

generating by said generating unit second image data using said plurality of acoustic signals; and

switching by said display unit from said display based on said first image data to a display based on said second image data in an identical position of said object.

According to the present invention, it is possible to provide an object information acquiring apparatus with which both an image display speed and an image resolution can be improved.

Further features of the present invention will become apparent from the following description of exemplary embodiments with reference to the attached drawings.

### BRIEF DESCRIPTION OF THE DRAWINGS

FIG. 1 is a view showing an overall configuration of an ultrasound diagnostic apparatus according to the present invention;

FIG. 2 is a view showing a configuration of an image generation unit according to a conventional example;

FIG. 3 is a view showing configurations of an image generation unit and an image storage unit according to a first embodiment;

FIG. 4 is a view showing a configuration of a delay-and-sum circuit;

FIG. 5 is a view showing the configuration of the image storage unit;

FIG. 6 is a view showing a configuration of an addition calculation circuit;

FIGS. 7A and 7B are views showing mechanical scanning performed by an ultrasound probe;

FIGS. 8A to 8D are views showing a principle of a synthetic aperture method;

FIG. 9 is a view showing the synthetic aperture method applied to a slice surface;

FIGS. 10A and 10B are views showing an output timing of a slice image;

FIGS. 11A and 11B are views showing a method of generating a three-dimensional ultrasound image;

FIG. 12 is a view showing configurations of an image generation unit and an image storage unit according to a second embodiment;

FIG. 13 is a view showing a control flow of an adaptive calculation circuit;

FIG. 14 is a view showing a control flow of a referral signal synthesis block;

FIGS. 15A and 15B are views showing a method of generating a three-dimensional ultrasound image according to the second embodiment; and

FIGS. 16A and 16B are views showing a method of generating a three-dimensional ultrasound image according to a third embodiment.

### DESCRIPTION OF THE EMBODIMENTS

Respective configurations of the present invention will be described in further detail below with reference to the drawings.

An object information acquiring apparatus according to the present invention uses a technique of obtaining object information in the form of image data by transmitting an acoustic wave to an object and receiving an acoustic wave (an echo signal) reflected in the object interior. An acoustic wave is a type of elastic wave, typically an ultrasound wave but also including elastic waves known as sound waves and ultrasound waves. A probe receives the acoustic wave propagating from the object interior. As described above, the information obtained from the object interior reflects differences in the acoustic impedance of tissue in the object interior.

An ultrasound diagnostic apparatus that performs a diagnosis on an object such as an organism will be described below as a representative example of the object information acquiring apparatus.

### First Embodiment

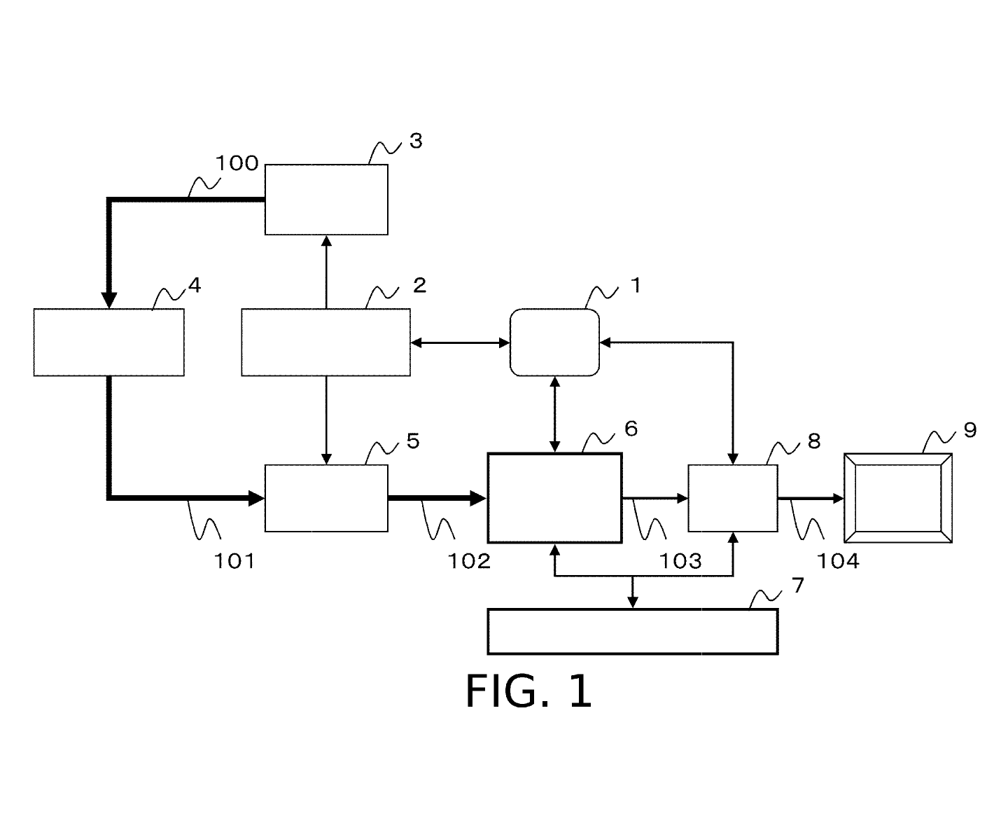

FIG. 1 is a view showing an overall configuration of the ultrasound diagnostic apparatus according to the present invention. First, to describe the overall configuration of the ultrasound diagnostic apparatus, main control of an apparatus main body is performed by an MPU (microprocessor unit) 1 such that a series of transmission/reception operations is performed by an ultrasound probe 4 that is connected to a transmission unit 3 and a reception unit 5 controlled by a transmission/reception control unit 2. The ultrasound probe 4 is used in contact with either the object or a holding member or the like holding the object, whereby ultrasound waves are transmitted to and received from the object. The ultrasound probe 4 is constituted by a plurality of transducers that transmit an ultrasound wave on the basis of a transmission analog signal 100 serving as an applied drive signal, receive a propagating ultrasound wave, and output a reception analog signal 101. The ultrasound probe 4 has an N-channel transducer array constituted by a linear array or a two-dimensional array. The transducer array may be any array that performs electronic scanning on a tomographic slice surface using an ultrasound beam in order to create a normal B mode ultrasound image. More specifically, a 1 D, 1.5 D, or 1.75 D transducer array may be used. A transducer array having a 2 D configuration may also be used as long as it is capable of generating an image by scanning a two-dimensional cross-section using electronic scanning. Linear scanning, in which an ultrasound beam scans a surface electronically by performing a substantially parallel motion, or the like is used as the electronic scan performed by the ultrasound probe 4. Linear scanning is advantaged in that an imaging width generated by the electronic scan is fixed, a wide imaging area is obtained even in parts close to the ultrasound probe 4, a lateral direction resolution is not dependent on an imaging depth (a depth measured from a joint surface between the probe and an imaging subject), and so on.

The ultrasound probe 4 is constituted by an oscillator in which electrodes are formed on either end of a piezoelectric ceramic, represented by PZT, or a piezoelectric material (a piezoelectric body) such as a polymer piezoelectric element, represented by PVDF, for example. Note that PZT is lead zirconium titanate, and PVDF is polyvinylidine difluoride. When the pulse-form or continuous wave-form transmission analog signal 100 is applied to the electrodes of the oscillator, the piezoelectric body expands and contracts. As a result of this expansion and contraction, pulse-form or continuous wave-form ultrasound waves are generated from the respective transducers, and by synthesizing these ultrasound waves, a transmission beam is formed. The respective transducers also expand and contract upon reception of propagating ultrasound waves, and generate electric signals as a result. These electric signals are output as the ultrasound wave reception analog signal 101. Here, elements employing different conversion methods may be used as the transducers. For example, the aforesaid oscillator may be used as the elements that transmit the ultrasound waves, while transducers employing a light detection method may be used as the elements that receive the ultrasound waves. A transducer employing a light detection method detects an ultrasound wave by converting the ultrasound wave into an optical signal, and is constituted by a Fabry-Perot resonator or a Fiber Bragg grating, for example.

The transmission/reception control unit 2 is controlled by software of the MPU 1 to control the transmission unit 3 and the reception unit 5 respectively on the basis of commands and information from an input operation unit. The transmission unit 3 is constituted by a pulsar drive circuit that supplies N channels of transducers constituting the ultrasound probe 4 with a number of transmission analog signals 100 corresponding to the N channels. The reception unit 5 first implements analog amplification processing on the weak reception analog signals 101 output from the N channels of transducers using a first stage LNA amplifier. Next, the reception unit 5 implements further analog amplification processing using a TGC (Time Gain Compensation) amplifier. Signals in an unnecessary frequency band are cut from the output of this amplifier using an AAF (Anti Aliasing Filter), whereupon A/D conversion processing is performed on each channel using a high-speed sampling (CLOCK) A/D converter. As a result, N channels of echo detection data 102 converted into reception digital signals are output.

An image generation unit 6 outputs two-dimensional image data 103 known as a B mode image by executing phase alignment processing, signal processing, and image generation on the input echo detection data 102. A DSC (Digital Scan Converter) 8 serves as display control unit for writing the input two-dimensional image data 103 (first image data, second image data, or the like) temporarily to an image storage unit 7 and outputting the two-dimensional image data 103 in the form of a video signal 104 in alignment with a timing of a horizontal synchronization frequency. A display unit 9 displays the B mode image upon input of the video signal 104.

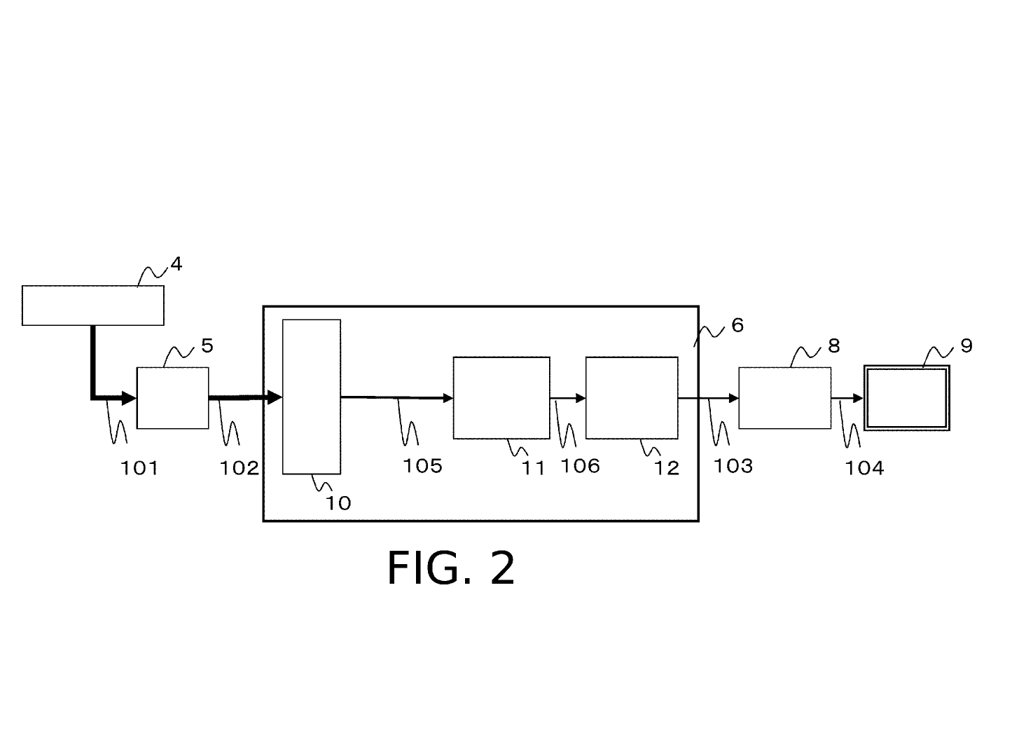

FIG. 2 is a view showing a configuration of the image generation unit 6 according to a conventional example. The N channels of echo detection data 102 output from the reception unit 5 are subjected to lateral direction phase alignment processing, which is a basic function of reception focus processing, by a delay-and-sum circuit 10 and output as added RAW data 105. In other words, the delay-and-sum circuit 10 performs delay-and-sum processing on the RAW data 105 using the plurality of electric signals (the echo detection data) and outputs a plurality of acoustic signals. The lateral direction is a direction in which an electronic scan is performed using a plurality of elements, and in a lateral array probe corresponds to the array direction of the plurality of elements. The lateral direction corresponds to a first direction according to the present invention. One-dimensional display data 106 are generated in a signal processing circuit 11 by implementing signal processing such as envelope detection or STC (Sensitivity Time Gain Control) on the RAW data 105. The one-dimensional display data 106 are single scanning line unit display data known as an A mode image. The image processing circuit 12 outputs the two-dimensional image data 103 known as the B mode image by converting the A mode image into a tomographic slice image constituted by two-dimensional data while successively storing A mode images in scanning line units. The two-dimensional image data constituting the tomographic image correspond to first image data according to the present invention. That is, a plurality of the first image data is constructed along with the second direction, by use of a plurality of acoustic signals.

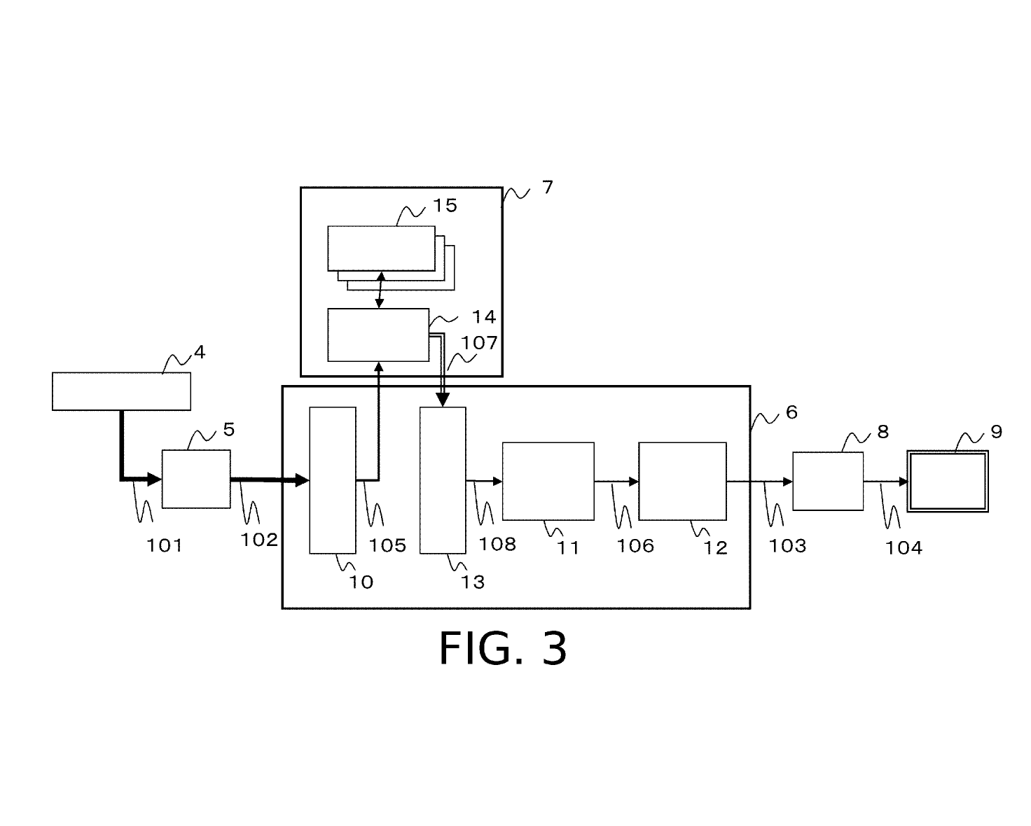

FIG. 3 is a view showing internal configurations of the image generation unit 6 and the image storage unit 7 according to the first embodiment of the present invention. In comparison with the configuration of the conventional example shown in FIG. 2, new blocks representing an image memory 15, a memory control circuit 14, and an addition calculation circuit 13 have been added. The image memory 15 is a location for temporarily storing plural RAW data 107, while the memory control circuit 14 controls reading/writing memory areas of the image memory 15. When these blocks are provided, a processing method of the RAW data 105 generated by the delay-and-sum circuit 10 is different. More specifically, the addition calculation circuit 13 is operated to reference the stored plural RAW data 107 simultaneously. The tomographic slice image data serving as the first image data are generated by similar processing to that of FIG. 2, i.e. using the RAW data 105 without passing through the addition calculation circuit 13.

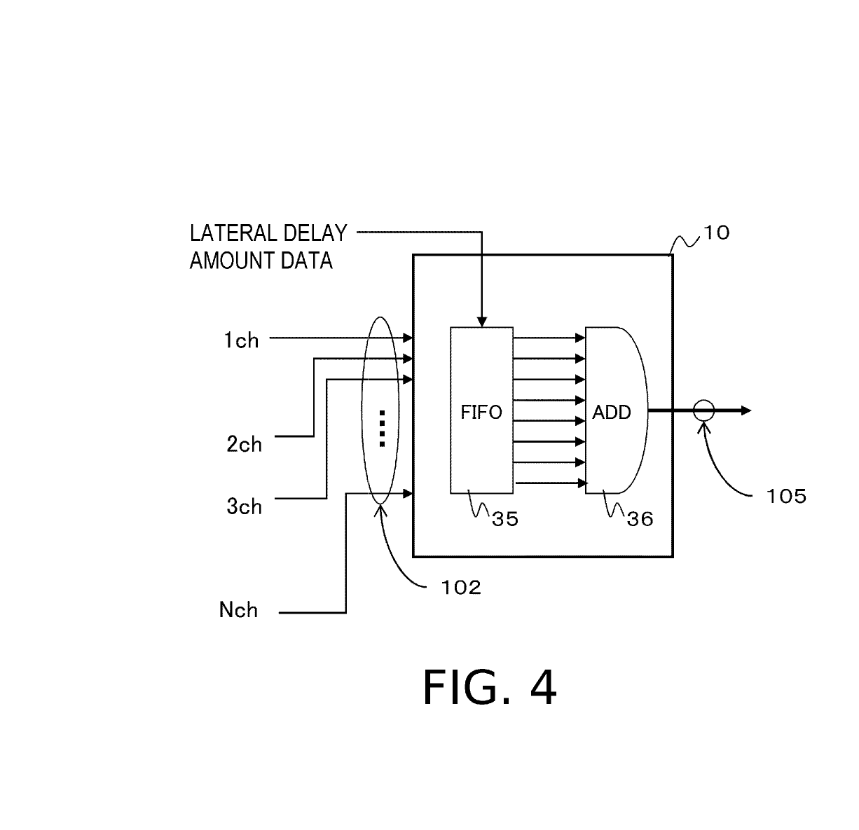

Operations of the respective constituent elements will now be described in further detail. FIG. 4 is a view showing the internal configuration of the delay-and-sum circuit 10 for the lateral direction. This circuit performs delay-and-sum processing for phase-aligning the echo detection data 102 output from the A/D converter 34, or in other words reception beam focus addition processing. To obtain an appropriate focus delay time for lateral delay amount data provided from the MPU 1, a desired focus delay is applied to the N channels of echo detection data 102 using a FIFO memory 35, whereupon an addition calculation is performed on all of the N channels by an addition calculator 36. As a result, phase-aligned RAW data 105 representing a plurality of acoustic signals obtained along a desired scanning line (signals corresponding to intensities of acoustic waves in respective positions of the object interior). In other words, the delay-and-sum circuit 10 performs delay-and-sum processing on the RAW data 105 using the plurality of electric signals (the echo detection data) and outputs a plurality of acoustic signals.

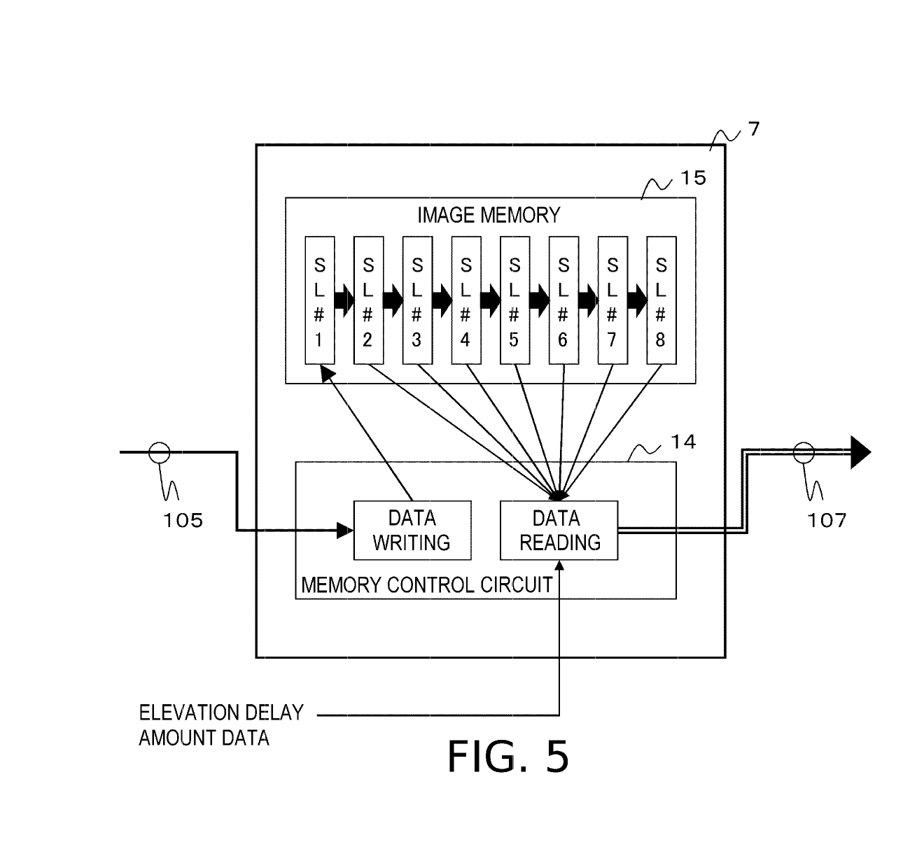

FIG. 5 is a view showing an example of an internal configuration of the image storage unit 7. The image memory 15 according to this embodiment is configured to be capable of storing data corresponding to eight slices. First, at a timing when one slice of RAW data is written to SL#1, sequential data shifting operations are performed in slice units such that the slice data in SL#1 are moved to SL#2, the slice data in SL#2 are moved to SL#3, and so on. Eventually, eight slices of data can be stored simultaneously from SL#1 to SL#8. Of SL#1 to SL#8, SL#1 operates as a write only memory area, while SL#2 to SL#8 operate as read only memory areas. The memory control circuit 14, meanwhile, is a control circuit that performs write address control and read address control non-synchronously. Here, the write address control is control for writing the RAW data 105 to a memory address corresponding to SL#1 of the image memory 15. Further, the read address control is control for reading the plural RAW data 107 at a memory address corresponding to the elevation delay amount data designated by the MPU 1 simultaneously from the seven slices of data stored in SL#2 to SL#8 stored in the image memory 15.

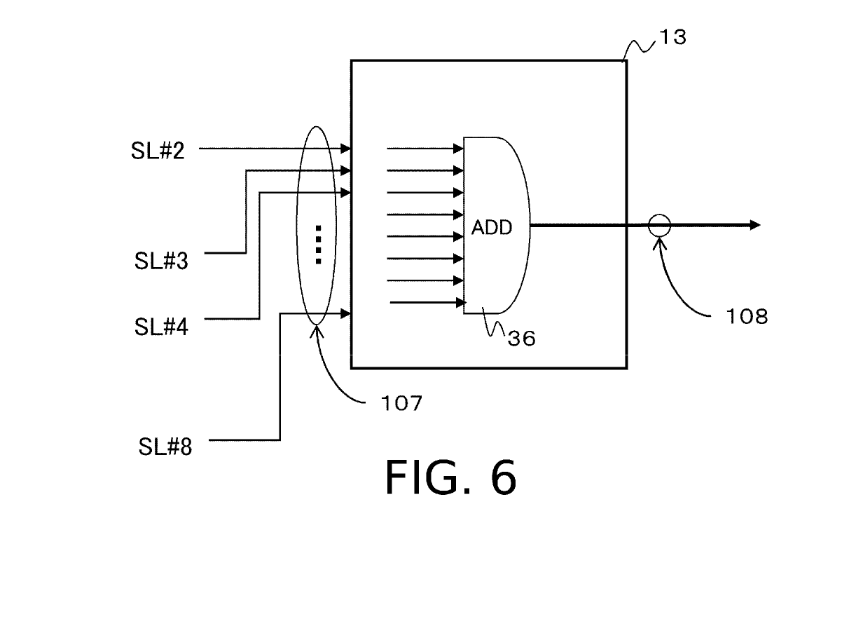

FIG. 6 is a view showing an internal configuration of the addition calculation circuit 13, which is a circuit that performs addition processing on the plural RAW data 107 output in accordance with the elevation delay amount data designated by the MPU 1 such that added RAW data 108 to which a synthetic aperture method has been applied in the elevation direction are output. The elevation direction is orthogonal to and intersects the lateral direction, and corresponds to a second direction according to the present invention. The difference between the addition calculation circuit 13 and the delay-and-sum circuit 10 that performs the lateral direction phase alignment processing is that elevation direction phase control is performed by the memory control circuit 14, and therefore the addition calculation circuit 13 only performs addition processing on the data without the need for FIFO.

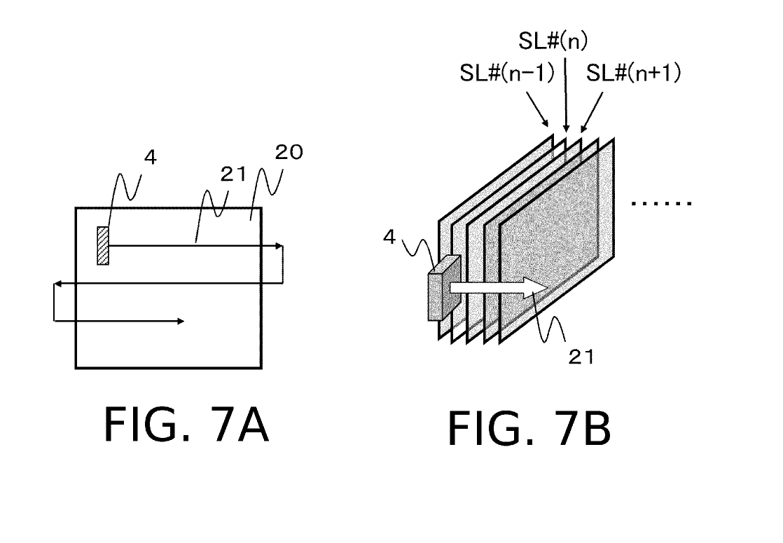

FIG. 7A is a view showing an operation for obtaining a three-dimensional ultrasound image of a wide scanning area 20 by moving the linear array ultrasound probe 4 mechanically along an elevation direction movement path 21. By moving the ultrasound probe 4 at a constant speed such that the tomographic slice image described above is obtained repeatedly in each position of the movement path 21, and then arranging the obtained tomographic slice images closely, a three-dimensional ultrasound image of an entire examination area can be formed.

FIG. 7B shows a scanning procedure performed when the linear array probe 4 obtains tomographic slice images in order of SL#(n−1), SL#(n), SL#(n+1) while moving continuously along the elevation direction movement path 21. Two-dimensional image data constituting the respective tomographic slice images are output at intervals of a fixed period relative to the elevation direction. At this time, the ultrasound probe 4 may be moved intermittently or continuously. When the ultrasound probe 4 is moved continuously, the tomographic slice images are not strictly orthogonal to the movement direction. However, it is assumed here for ease of description that the tomographic slice images are orthogonal to the movement direction.

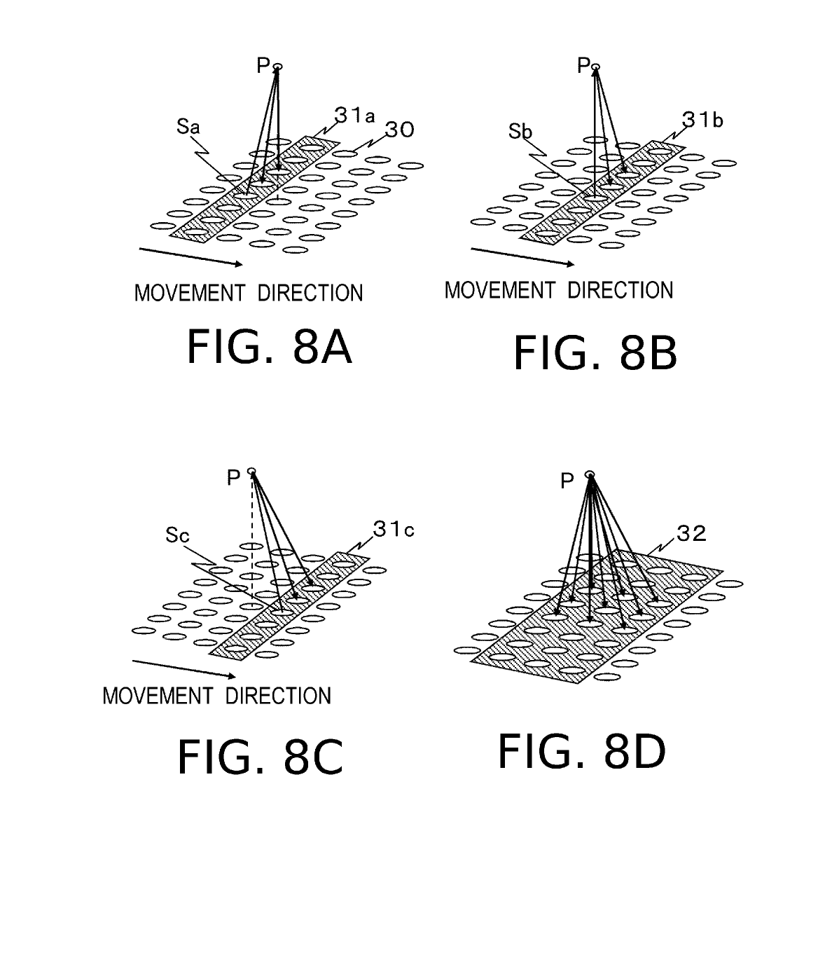

FIG. 8 is a view illustrating a principle of the synthetic aperture method, which is an image reconstruction method serving as background to the present invention. Small arranged elliptical graphics 30 denote positions of the respective transmission/reception elements during scanning of the respective slice surfaces, and points P denote desired focus points within a three-dimensional space. FIG. 8A shows a point at which an (n−1)th slice surface is scanned by a transmission/reception element group surrounded by a rectangular graphic 31a, wherein a part of an ultrasound beam emitted from a central portion Sa also propagates in a P point direction and a reflection wave thereof is received by the transmission/reception elements in positions within the rectangular graphic 31a. FIG. 8B shows a condition in which the probe moves to the position of an nth slice such that a transmission/reception element group surrounded by a rectangular graphic 31b emits another ultrasound beam from a central portion Sb, a part of which also propagates in the P point direction such that a reflection wave thereof is received by the transmission/reception element group in positions within the rectangular graphic 31b. FIG. 8C shows a condition in which the probe moves to the position of an (n+1)th slice such that a transmission/reception element group surrounded by a rectangular graphic 31c emits another ultrasound beam from a central portion Sc, a part of which also propagates in the P point direction such that a reflection wave thereof is received by the transmission/reception element group in positions within the rectangular graphic 31c.

The transmission/reception timings on the respective slice surfaces differ from each other. Here, a time from transmission to reception is calculated from a propagation distance and an acoustic velocity in order to adjust a reception time of the signals to be added in each reception element, whereupon reflection signals from identical P points are added together. In so doing, as shown in FIG. 8D, it is possible to obtain an equivalent result to that obtained in a case where signals received by a virtual two-dimensional probe constituted by a transmission/reception element group positioned within a rectangular graphic 32 are calculated by two-dimensional delay-and-sum processing. As a result, an ultrasound image having a similar resolution to that obtained with a two-dimensional array ultrasound probe can be obtained with the linear array ultrasound probe, and a particular improvement can be achieved in the elevation direction resolution. A method of obtaining an equivalent resolution to that of a case in which a reception aperture is substantially increased by synthesizing reception signals having different ultrasound wave emission times is known as a synthetic aperture method. In this embodiment, data obtained using a synthetic aperture correspond to second image data.

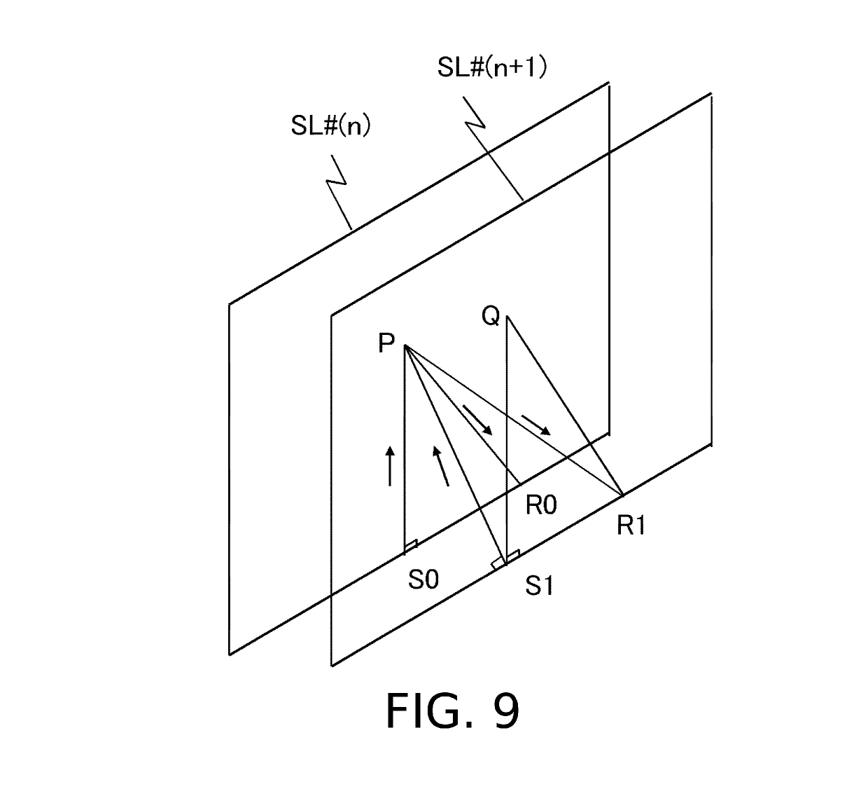

FIG. 9 is a view illustrating the synthetic aperture method applied to a slice surface for the purpose of image reconstruction according to the first embodiment. To facilitate description, the focus point P is assumed to be in a plane of a slice surface SL#(n). An ultrasound beam emitted in a perpendicular direction from a center S0 of a transmission/reception element group is reflected at the P point and received by a transmission/reception element in an R0 position. Next, the probe moves to a position of a slice surface SL#(n+1), whereupon another ultrasound beam is emitted from a position S1 corresponding to S0. Although the ultrasound beam is emitted in a perpendicular direction, a part thereof also propagates in the direction of the P point within the tomographic slice surface SL#(n) such that the ultrasound wave reflected by the P point is received at a point R1 corresponding to the R0 point. The delay-and-sum of the synthetic aperture method described above can be realized by adding together the reception signal at R0 and the reception signal at R1 after adjusting a deviation between reception times thereof corresponding to respective propagation times from emission to reception via reflection by the P point.

Next, a point Q having an identical distance from a point S1 to the P point in the perpendicular direction in the slice surface SL#(n+1) will be considered. In this case, a triangle formed by the points S1, P, R1 and a triangle formed by the points S1, Q, R1 are clearly congruent, and therefore a time required to reach R1 from S1 via the P point is identical to a time required to reach R1 from S1 via the Q point. This relationship is identical in relation not only to the transmission/reception element in the R1 position, but also to the other reception elements in the same transmission/reception element group, and therefore, in positions of the slice surface SL#(n+1), identical added signals are obtained from a linear delay-and-sum result focusing on the P point and a delay-and-sum result focusing on the Q point. Hence, in the two-dimensional delay-and-sum processing performed in relation to the P point, linear delay-and-sum processing may be performed first on each slice surface to determine the delay-and-sum signals of the P point and the Q point, whereupon appropriate linear delay-and-sum processing is performed in the elevation direction to add together the delay-and-sum signals of the P point and the Q point.

More specifically, the image generation unit 6 according to the first embodiment, shown in FIG. 3, realizes this principle through real time processing in which the RAW data 105 output from the delay-and-sum circuit 10 are stored temporarily in the image memory 15 via the memory control circuit 14. In the addition calculation circuit 13, RAW data having a corresponding delay amount from the stored plural RAW data 107 are referenced via the memory control circuit 14, whereupon elevation direction delay-and-sum processing is executed on the basis of the synthetic aperture method. As a result, an equivalent effect to that obtained when the two-dimensional delay-and-sum processing shown in FIG. 8 is executed can be obtained with a smaller circuit scale.

When the synthetic aperture method shown in FIG. 9 is used, as a basic principle, the data precision following the delay-and-sum processing improves as the number of referenced slice surfaces increases. More specifically, a first requirement for improving the resolution is to secure as large an area of the image storage unit 7 as possible for the image memory 15 that stores the RAW data 105 output from the delay-and-sum circuit 10. A further requirement is to achieve a speed increase so that the delay-and-sum circuit 13 can perform the synthetic aperture processing using a large amount of the plural RAW data 107 simultaneously. For this purpose, the respective circuit scales of the two circuits must be combined.

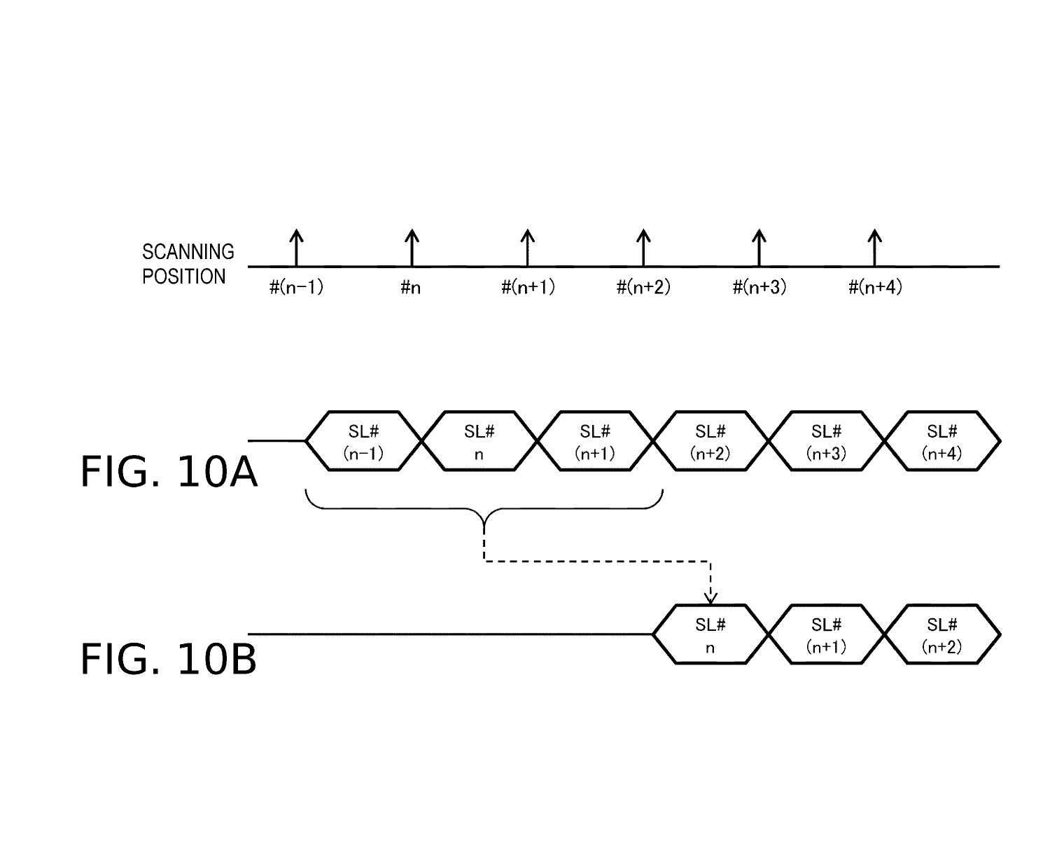

FIG. 10 is a view showing timings at which the two-dimensional image data 103 generated by the image generation unit 6 are output in relation to scanning positions of the ultrasound probe 4. As shown by the output timings in FIG. 10A, in the conventional image generation unit 6, there is no time delay in image generation from acquisition of the echo detection data 102 to generation of the two-dimensional image data 103. Therefore, tomographic slice images SL#(n−1) to SL#(n+4) are output in real time relative to scanning positions #(n−1) to #(n+4) of the ultrasound probe 4.

At the output timings according to the first embodiment, shown in FIG. 10B, on the other hand, three tomographic slice images are stored temporarily in the image storage unit 7, and the two-dimensional image data 103 are generated by implementing addition calculation processing through a synthetic aperture using the three tomographic slice images. Therefore, a wait time is generated up to a point at which the plurality of tomographic slice images required for the synthetic aperture are collected. More specifically, in the example shown in FIG. 10B, when a fourth slice SL#(n+2) is taken, synthetic aperture processing is implemented using the stored tomographic slice images SL#(n−1), SL#(n), SL#(n+1). As a result, a shift in the output timing occurs such that the two-dimensional image data 103 of SL#1 are generated at the stage where the scanning position of the ultrasound probe 4 reaches #(n+2).

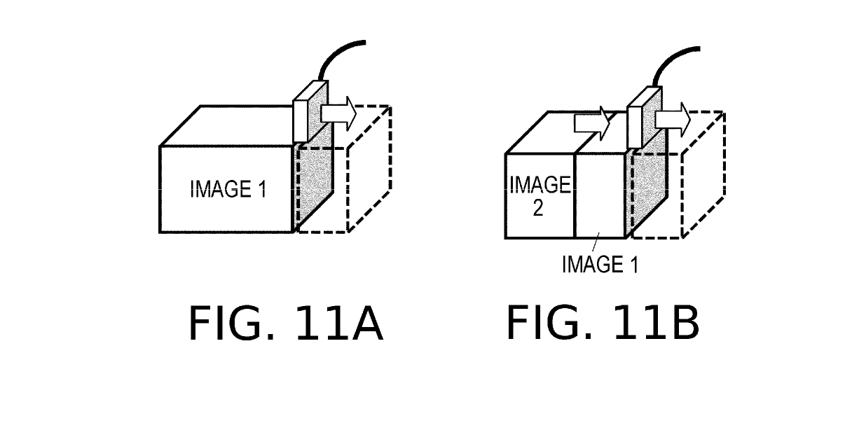

FIG. 11 is a view showing ultrasound images displayed in real time by the image display unit 9. The effect of the output timing shift described above will now be described. At a conventional output timing shown in FIG. 11A, tomographic slice images are generated in accordance with the scanning position of the ultrasound probe 4, whereby an area of a three-dimensional ultrasound image 1 displayed by overlapping the slice images is generated in synchronization with the movement of the probe.

At the output timing according to this embodiment, shown in FIG. 11B, the time delay for performing synthetic aperture processing is generated relative to the scanning position of the ultrasound probe 4, and therefore an image 2 is displayed at a delay relative to the scanning position. At this time, the image 1, i.e. the conventional tomographic slice image, may be displayed in real time in accordance with the scanning position, and when image generation using the synthetic aperture is complete, the image 1 may be switched to the image 2. In so doing, the tomographic slice images can be presented immediately after the scan without a wait time, and when calculation of the synthetic aperture is complete, an image having a higher resolution can be presented. As a result, real time image display and high-precision image display can both be achieved. In other words, an improvement in image resolution can be achieved while maintaining the image display speed.

### Second Embodiment

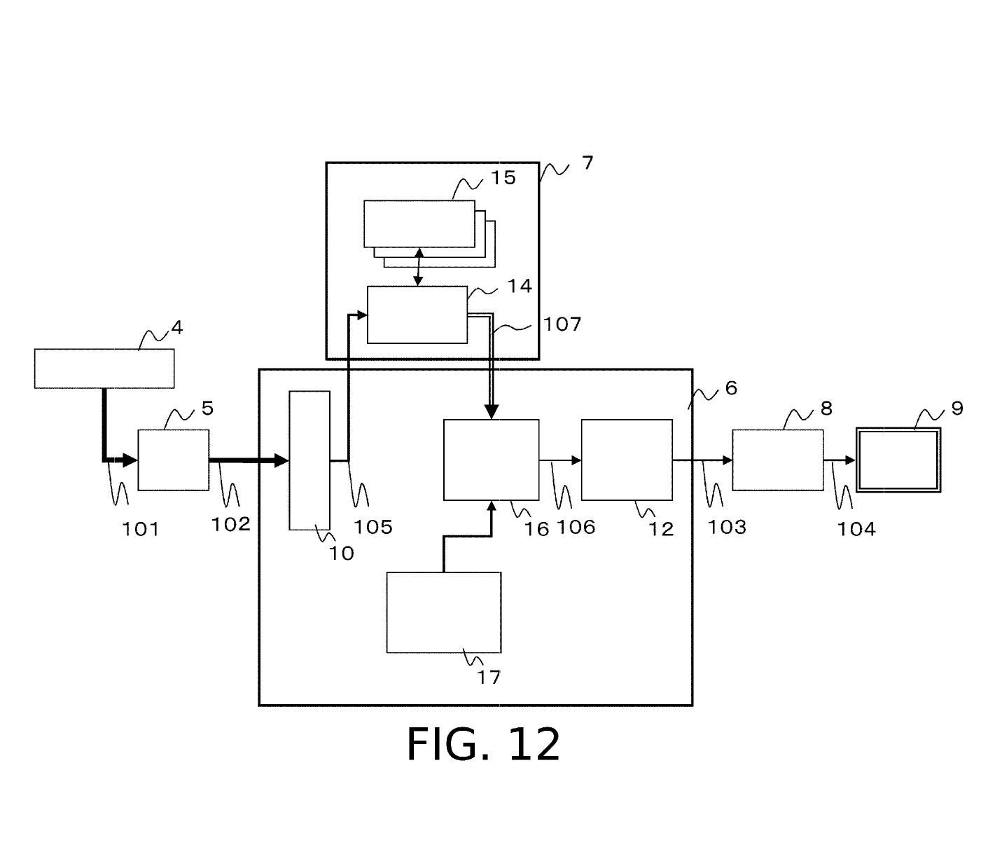

FIG. 12 is a view showing an internal configuration of the image generation unit 6 and the image storage unit 7 according to a second embodiment of the present invention. In comparison with the configuration according to the first embodiment, shown in FIG. 3, new blocks constituting an adaptive calculation circuit 16 and a referral signal synthesis block 17 are added in place of the addition calculation circuit 13 and the signal processing circuit 11 such that adaptive signal processing is implemented by referencing the stored plural RAW data 107. Note, however, that the data of the tomographic slice images constituting the first image data are generated by similar processing to FIG. 2, i.e. using the RAW data 105 without passing through the adaptive calculation circuit 16.

Here, an outline of an operation performed during adaptive signal processing will be described. Adaptive signal processing is known in the field of radar as a method of estimating a target distance with a high degree of precision. Proc. Acoustics, Speech Signal Process., pp. 489-492 (March 2005) describes a method of improving resolution by employing adaptive signal processing when generating ultrasound echo image data. Further, a method in which both frequency domain interferometry (FDI) and adaptive signal processing are performed is known as a technique for improving spatial resolution in a depth direction. Conf Proc IEEE Eng Med Biol Soc. 2010; 1: 5298-5301 and Japanese Patent Application Publication No. 2010-183979 disclose results of an operation for forming an image of a layer structure of a blood vessel wall by performing an FDI method and a CAPON method, which is a type of adaptive signal processing, using electric signals output by a probe.

In adaptive signal processing, a processing parameter is varied adaptively in accordance with a reception signal. The CAPON method, which is a type of adaptive signal processing, is a method of processing a plurality of input signals such that in a condition where sensitivity to a focus position is fixed, power is minimized.

The FDI method is a method of analyzing reception signals (electric signals output from a probe) at each frequency, and estimating a reception power in a focus position using phase information relating to a plurality of frequency components. When a plurality of frequencies that are phase-aligned in a certain reference position are considered, a product of a distance from the reference position and a wave number is found to be proportionate to an amount of variation in the phase. In other words, when a certain focus distance is set and both the distance from the reference position to the focus distance and the frequency, or in other words the wave number, are known, it is possible to calculate a degree of phase variation. By applying the degree of phase variation to reception signals of respective frequencies and adding the reception signals together, the reception power at the focus distance can be estimated.

By combining the FDI method with adaptive signal processing, the reception power in the focus position can be estimated not based on a phase variation amount/weighting determined in advance in relation to the reception signals analyzed at each frequency component, but based on a phase variation amount/weighting calculated in accordance with the signals using adaptive signal processing.

In the present invention, the adaptive signal processing is not limited to the CAPON method, and a MUSIC method, an ESPRIT method, and so on may be used instead.

As described above, a typical ultrasound diagnostic apparatus forms an image by obtaining an envelope of a received waveform. When the FDI method and the CAPON method are applied in this case to improve the resolution further, it is envisaged that a plurality of reflection layers will exist in the FDI processing range. In an atmospheric observation radar, correlation between a plurality of reflection waves from the plurality of reflection layers can be suppressed by making an observation time sufficiently long, but during medical ultrasound imaging, the observation time of a single processing range is short, and therefore correlation between the plurality of reflection waves cannot be suppressed. A plurality of reflection waves from close reflection layers are therefore considered to have a high correlation.

It is known that when adaptive signal processing such as the CAPON method or the MUSIC method is applied as is to a plurality of reflection waveforms having a high correlation, unintended operations such as canceling out of a desired signal occur. By applying a frequency averaging method in this situation, operations of the FDI method and the CAPON method can be checked, and therefore a frequency averaging method is preferably used when FDI and CAPON are applied to medical ultrasound imaging.

According to Japanese Patent Application Publication No. 2010-183979, even when an observation subject having a different frequency characteristic exists, a calculation referral signal that takes the frequency characteristic of the subject into account can be generated by synthesizing reference signals. As a result, an improvement in spatial resolution in the depth direction can be achieved through adaptive signal processing.

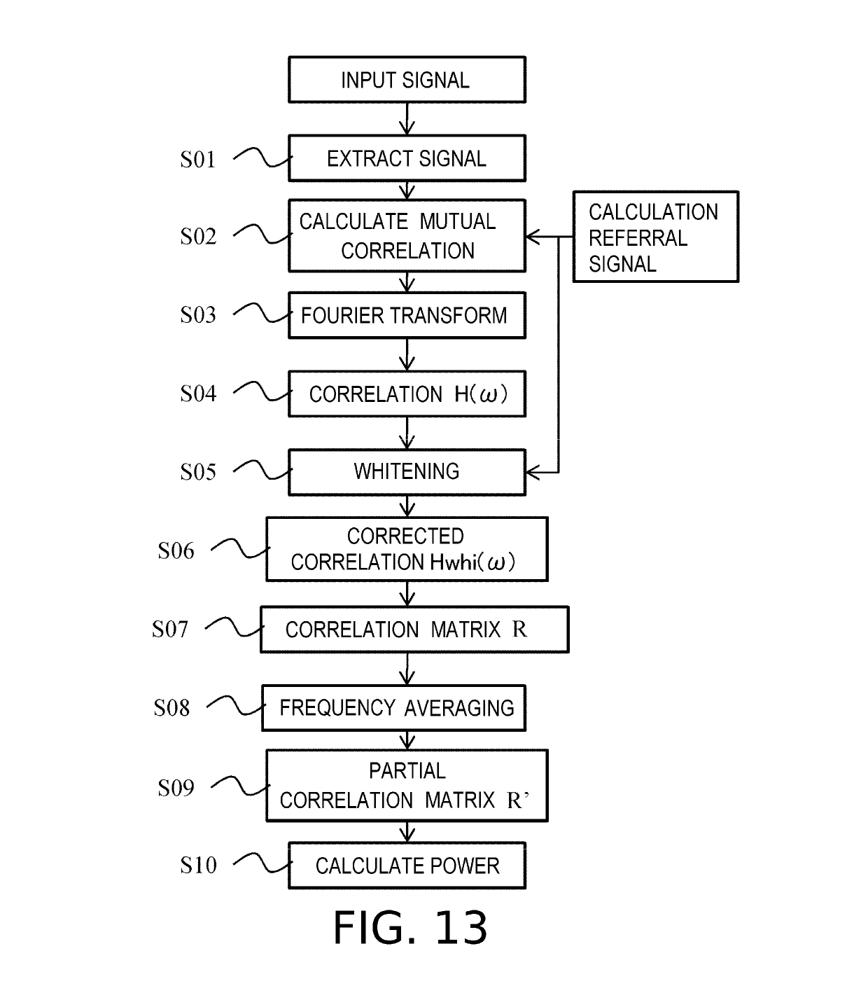

FIG. 13 is a flowchart illustrating processing performed by the adaptive calculation circuit 16. The adaptive calculation circuit 16 extracts signals within a single processing period, or in other words signals within the processing range, from image signals input from the image storage unit 7 (S01). Next, a mutual correlation between a plurality of calculation referral signals input from the referral signal synthesis block 17 is calculated (S02). Here, processing performed on one of the plurality of calculation referral signals is illustrated as an example, but in actuality, similar processing is performed on the plurality of input calculation referral signals. A correlation H (ω) for each frequency is then determined by subjecting the mutual correlation to Fourier transform (S03, S04).

Next, whitening processing is performed using the calculation referral signal, as shown in Equation 1 (S05). When the referral signal is g (t) and the Fourier transform implemented thereon is G (ω), whitening can be performed as shown in Equation (1), and as a result, a corrected correlation Hwhi (ω), which is a signal having a flattened frequency spectrum, can be calculated (S06). Note that η denotes noise power.

[Math. 1]

H_{whi}(ω)=H(ω)/(|G(ω)|²+η) (1)

Next, frequency domain interferometry and frequency averaging are applied to the flattened signal. More specifically, a correlation matrix R having i, j components is formed, as shown in Equation (2) (S07).

[Math. 2]

rᵢⱼ=H_{whi}(ωᵢ)H_{whi}(ωⱼ)^{H} (2)

Next, a partial correlation matrix R′ is calculated using frequency averaging, as shown in Equation (3) (S08, S09). A depth direction power distribution P (r) is then estimated using the partial correlation matrix R′ thus determined (S10). Here, C is a constraint vector relative to a focus depth r, and kn is a wave number corresponding to an nth frequency. As a result of the processing described above, a plurality of depth direction power distributions corresponding to the plurality of calculation referral signals are calculated.

[Math. 3]

P(r)=1/(C^{H}R′⁻¹C)

C=[e^{jk}^{₁}^{r}, . . . , e^{jk}^{_{K}}^{r}] (3)

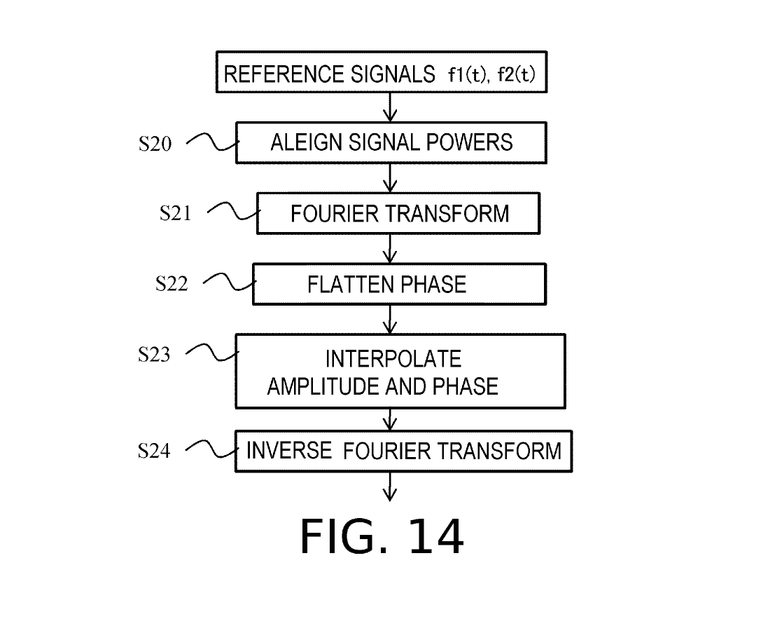

FIG. 14 is a flowchart illustrating processing performed by the referral signal synthesis block 17. Here, an example in which a calculation referral signal is created by interpolating (synthesizing) two reference signals f1 (t), f2 (t) will be described. Note that the reference signals used to create the calculation referral signals are stored in advance in the memory of the apparatus. First, the referral signal synthesis block 17 aligns the power of the reference signals f1 (t), f2 (t) (S20).

Next, phases φ1 (f), φ2 (f) are determined by subjecting the two reference signals to Fourier transform (S21). An aopt for minimizing Σ(φ′(f)) 2 when φ′(f)=φ(f)−af is then searched for in relation to each reference signal. By determining the φ′(f) of the reference signals at aopt, the phase is flattened (S22).

Next, an amplitude and a phase are interpolated using a predetermined interpolation ratio (also referred to as an interpolation coefficient) α, as shown in Equation (4), whereby synthesized REF3 (f) is calculated (S23). Note that an interpolation ratio α is an arbitrary value that satisfies 0≦α≦1. Here, REF1 (f) and REF2 (f) are frequency components of f1 (t), f2(t) following power correction and phase correction, respectively.

[Math. 4]

REF₁(f)=k′₁exp(jφ′₁(f))

REF₂(f)=k′₂exp(jφ′₂(f))

REF₃(f)={(1−α)k′₁+αk′₂(f)}exp(j((1−α)φ′₁(f)+αφ′₂(f))) (4)

Finally, a waveform of the calculation referral signal is determined by subjecting REF3 (f) to inverse Fourier transform (S24). Note that amplitude correction is preferably performed at this time to ensure that the signal power is fixed. The referral signal synthesis block 17 determines a plurality of calculation referral signals corresponding to a plurality of interpolation ratios α by varying the interpolation ratio α, and outputs the plurality of calculation referral signals to the adaptive calculation circuit 16. Values and variation steps of the interpolation ratios α may be set appropriately.

When the adaptive signal processing described above is performed, a process up to image generation is complicated. As a result, the processing time up to image generation is further increased in comparison with the delay-and-sum processing of the synthetic aperture method used in the first embodiment, and therefore the output timing shift increases further. In this embodiment, data obtained as a result of the adaptive signal processing correspond to the second image data.

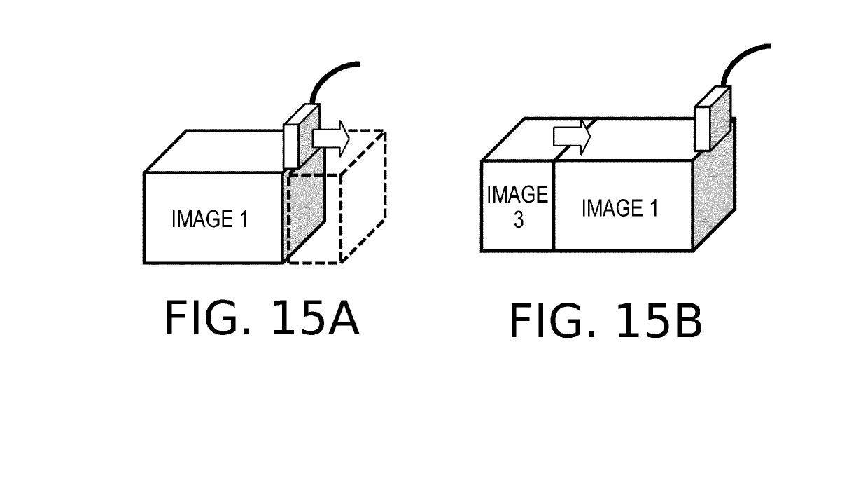

FIG. 15 is a view showing ultrasound images displayed in real time by the image display unit 9 according to the second embodiment. The effect of the output timing shift described above will now be described. At an output timing shown in FIG. 15A, tomographic slice images are generated in accordance with the scanning position of the ultrasound probe 4, whereby an area of the image 1 displayed by overlapping the slice images is generated in synchronization with the movement of the probe.

At an output timing shown in FIG. 15B, a time delay for performing adaptive signal processing is generated relative to the scanning position of the ultrasound probe 4, and therefore an image 3 from the adaptive signal processing is displayed at a delay once scanning is complete. At this time, the image 1 of the conventional tomographic slice images may be displayed in real time in accordance with the scanning position, and when the image 3 from the adaptive signal processing is generated, the image 1 may be switched to the image 3. In so doing, the tomographic slice images can be presented immediately after the scan without a wait time, and when the calculations of the adaptive signal processing are complete, a high-precision image can be presented. As a result, real time image display and high-precision image display can both be achieved. In other words, an improvement in image resolution can be realized while maintaining the image display speed.

### Third Embodiment

In a third embodiment, a case in which calculation circuits of two systems, namely the synthetic aperture processing and the adaptive signal processing described in the first and second embodiments, are provided simultaneously will be described. As regards the output timing of the calculation circuits for the two systems, the processing time of the calculation circuit used for adaptive signal processing is longer than that of the calculation circuit for the synthetic aperture processing, which is determined by the data amount of a tomographic slice image taken in advance, and therefore a time deviation occurs between image generation and output.

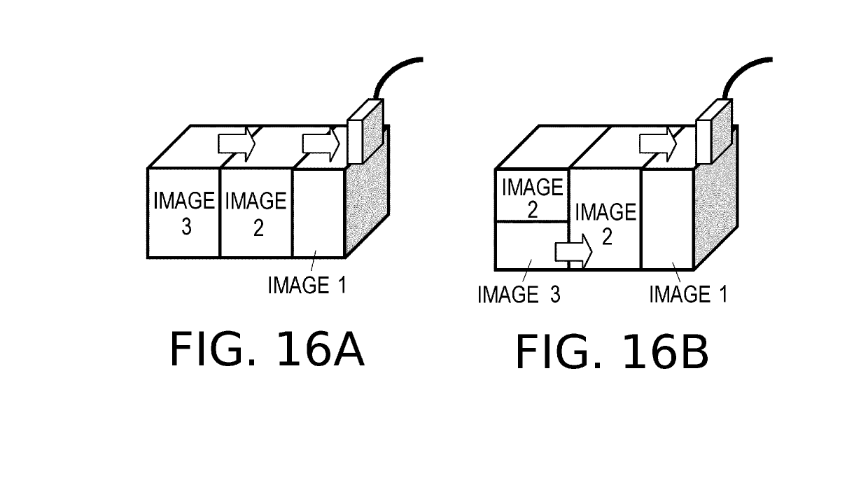

FIG. 16 is a view showing ultrasound images displayed in real time by the image display unit 9 according to the third embodiment. The effect of the output timing deviation between the two systems will now be described. At an output timing shown in FIG. 16A, tomographic slice images are generated in accordance with the scanning position of the ultrasound probe 4, whereby the area of the image 1 is generated in synchronization with the movement of the probe. Thereafter, the display is switched to the image 2 generated by the synthetic aperture processing, and at a further delay, the display is switched to the image 3 generated by the adaptive signal processing.

FIG. 16B shows a case in which the output timings of the image 1 and the image 2 are identical to FIG. 16A, but the position of three-dimensional data for displaying the image 3 is area-limited. For example, when adaptive signal processing is used, the resolution in a distance direction can be improved, but in parts close to the probe and so on, the image 2 formed from simple aperture control may have a higher resolution. In this case, by continuing to display the image 2 in a shallow part (a part having no more than a predetermined depth) and switching the display to the image 3 only in a deep part, a superior three-dimensional ultrasound image is obtained. At this time, the predetermined depth for switching between the image 2 and the image 3 may take a predetermined value obtained by experiment. In other words, the displayed image may be switched not only in time series but also by area. Further, in this embodiment, there are no limitations on a display combining method. In this embodiment, the data obtained by synthetic aperture processing correspond to the second image data, while the data obtained by the adaptive signal processing correspond to the third image data.

According to the embodiments described above, image generation can be performed in stages without impairing an image display speed in an ultrasound diagnostic apparatus that generates a three-dimensional ultrasound image using tomographic slice images obtained while continuously moving a linear array probe in an elevation direction. Simultaneously, an improvement in spatial resolution in the elevation direction can be achieved.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2011-201931, filed on Sep. 15, 2011, and, Japanese Patent Application No. 2012-175738, filed on Aug. 8, 2012, which are hereby incorporated by reference herein in their entirety.

What is claimed is:

1. An object information acquiring apparatus comprising:
a probe in which a plurality of elements that receive acoustic waves propagating from an object and convert said acoustic waves into electric signals are arranged in at least a first direction;
a scanning unit that moves said probe in a second direction that intersects said first direction;
a generating unit that determines intensities of said acoustic waves in respective positions of an object interior using said plurality of electric signals, generates a plurality of first image data corresponding to tomographic images of said object in said second direction using a plurality of acoustic signals based on said intensities, and generates second image data using said plurality of acoustic signals; and
a display control unit into which said first image data and said second image data are input, and which displays on a display unit an image representing information relating to said object interior,
wherein said display control unit displays on said display unit a display based on said first image data, and
when said second image data are input from an identical position of said object, switches said display from said display based on said first image data to a display based on said second image data.

2. The object information acquiring apparatus according to claim 1, wherein said generating unit obtains said plurality of acoustic signals through delay-and-sum processing using said plurality of electric signals, and
generates said second image data through synthetic aperture processing using said plurality of acoustic signals.

3. The object information acquiring apparatus according to claim 1, wherein said generating unit obtains said plurality of acoustic signals through delay-and-sum processing using said plurality of electric signals, and
generates said second image data through adaptive signal processing using said plurality of acoustic signals.

4. An object information acquiring apparatus comprising:
a probe in which a plurality of elements that receive acoustic waves propagating from an object and convert said acoustic waves into electric signals are arranged in at least a first direction;
a scanning unit that moves said probe in a second direction that intersects said first direction;
a generating unit that determines intensities of said acoustic waves in respective positions of an object interior using said plurality of electric signals, generates a plurality of first image data corresponding to tomographic images of said object in said second direction using a plurality of acoustic signals based on said intensities, and generates second image data and third image data using said plurality of acoustic signals; and
a display control unit into which said first, second, and third image data are input, and which displays on a display unit an image representing information relating to said object interior,
wherein said display control unit displays on said display unit a display based on said second image data, and
when said third image data are input from an identical position of said object, switches said display from said display based on said second image data to a display based on said third image data.

5. The object information acquiring apparatus according to claim 4, wherein said generating unit obtains said plurality of acoustic signals through delay-and-sum processing using said plurality of electric signals,
generates said second image data through synthetic aperture processing using said plurality of acoustic signals, and
generates said third image data through adaptive signal processing using said plurality of acoustic signals.

6. The object information acquiring apparatus according to claim 4, wherein said display based on said second image data is displayed by said display unit, and said third image data are generated in an identical position of said object such that in a part of said object having not more than a predetermined depth, said display based on said second image data is displayed, and in a part that is deeper than said predetermined depth, said display based on said second image data is switched to a display based on said third image data.

7. A control method for an object information acquiring apparatus having:
a probe in which a plurality of elements that receive acoustic waves propagating from an object and convert said acoustic waves into electric signals are arranged in at least a first direction;
a scanning unit that moves said probe in a second direction that intersects said first direction;
a generating unit that determines intensities of said acoustic waves in respective positions of an object interior from said plurality of electric signals, and generates image data using a plurality of acoustic signals based on said intensities; and
a display unit that displays an image of said object based on said image data,
the control method comprising the steps of:
generating by said generating unit a plurality of first image data corresponding to tomographic images of said object in said second direction;
displaying on said display unit a display based on said first image data;
generating by said generating unit second image data using said plurality of acoustic signals; and
switching by said display unit from said display based on said first image data to a display based on said second image data in an identical position of said object.
